(19) United States
(12) Patent Application Publication
(10) Pub. No.: US 2026/0232018 A1
(43) Pub. Date: Aug. 13, 2026

(54) Vaporizer

(71) Applicant: Inspiranox Therapeutics Corporation, Dover, DE (US)

(72) Inventors: Nicolas Martinez Grosso, I, Montevideo (UY); Francisco Molinari, Montevideo (UY)

(21) Appl. No.: 19/536,680

(22) Filed: Feb. 11, 2026

(30) Foreign Application Priority Data
Feb. 11, 2025 (LU) LU103534

(51) Int. Cl. A24F 40/40; A24F 40/10; A24F 40/485

(52) U.S. Cl. CPC A24F 40/40; A24F 40/10; A24F 40/485

(57) ABSTRACT

A vaporizer for treatment of nicotine addiction and/or pulmonary conditions often associated with nicotine addiction such as chronic obstructive pulmonary disease (COPD) and pre-COPD conditions, e.g. chronic bronchitis. The vaporizer has a vapor chamber which contains a therapeutic solution. Ultrasonic cold vaporization creates a vapor of the solution. The vapor is heated by heating elements and discharged through a vapor outlet to a user. An air inlet provides ambient air into the chamber to prevent a vacuum lock.

### FIELD OF THE INVENTION

The present invention relates to a vaporizer and an electronic cigarette or vape for use in the treatment of nicotine addiction and/or pulmonary conditions often associated with nicotine addiction such as chronic obstructive pulmonary disease (COPD) and pre-COPD conditions, e.g. chronic bronchitis.

### BACKGROUND OF THE INVENTION

Electric or electronic vaporizers having a form and dimensions of a cigarette are known in the form of at least since 1930 (U.S. Pat. No. 1,775,947 A, issued 16 Sep. 1930). Gilbert (U.S. Pat. No. 3,200,819 A, issued 17 Aug. 1965) devised a smokeless non-tobacco electric cigarette.

Current electronic vaporizers for generating a vapor for inhalation in the form of a more or less cigarette-like article (also known and from time to time denoted herein as “e-cigarette” or “vape”) use a resistance coil as the heating element for generating vapor from a liquid (often referred to as “e-liquid”) usually containing a base formulation composed of propylene glycol and glycerin in admixture with a variety of flavors, nicotine and other additives. The vaporization occurs in direct contact of the liquid with the resistance coil generating temperatures of at least 100° C., typically of from 100° C. to 315° C.

Vapes are less harmful than tobacco cigarettes because no organic parts of plants are burned. There are indications that vapes help consumers in tobacco cessation more effectively in comparison to conventional nicotine replacement therapies. However, long-term studies of potential harmful effects are missing and there are indications that the high temperature of the resistance coil leads to a contamination of the aerosol with metal particles. Furthermore, the vapor generated from conventional e-liquids is often perceived as unpleasant by consumers, especially those who want to quit smoking, reducing compliance rate. Moreover, the high temperatures occurring in vaporization by conventional vapes make them incompatible with application of drugs such as active ingredients helping nicotine-addicted customers in the process of tobacco cessation or drugs used for treatment of condition often associated with nicotine addiction, in particular COPD.

### SUMMARY OF THE INVENTION

The present invention provides a novel vape system overcoming one or more of the deficiencies of prior art vaporizers.

In particular, the vaporizer of the present invention comprises:
a vapor chamber designed to contain vapor generated from a liquid inside the chamber under temperature controlled condition;
an air inlet designed for actively or passively introducing ambient air into the chamber under the condition that the pressure inside the chamber is lower than the ambient pressure outside the chamber;
a vapor outlet for discharging vapor generated in the chamber and directing the vapor to a user;
one or more means for cold vaporization of a liquid; and heating means for heating vapor generated in the chamber.

In another embodiment, the vaporizer of the present invention comprises:
a vapor chamber having a lower part and an upper part and sidewalls;
an aqueous solution having one or more additives contained in the lower part of the chamber;
one or more ultrasonic heads provided in the lower part of the vapor chamber for cold vaporization of the aqueous solution to form a vapor;
one or more electrical resistance heating elements provided on the sidewalls of the vapor chamber to heat vapor in the vapor chamber;
electronic control elements and a battery for activating and providing power to the one or more ultrasonic heads and the one or more electrical resistance heating elements;
a vapor outlet provided in the upper part of the vapor chamber for discharging vapor formed in the chamber and having a mouthpiece for directing the vapor to a user; and
an air inlet provided in the upper part of the vapor chamber having a one-way inlet valve and an elongate tube extending into the lower part of the vapor chamber for introducing ambient air into the chamber when pressure inside the chamber is lower than ambient air pressure outside the chamber.

### BRIEF DESCRIPTION OF THE DRAWINGS

FIG. 1 is a schematic cross-sectional view of a first embodiment of a vaporizer.

FIG. 2 is a schematic cross-sectional view of a second embodiment of a vaporizer.

### DETAILED DESCRIPTION OF THE INVENTION

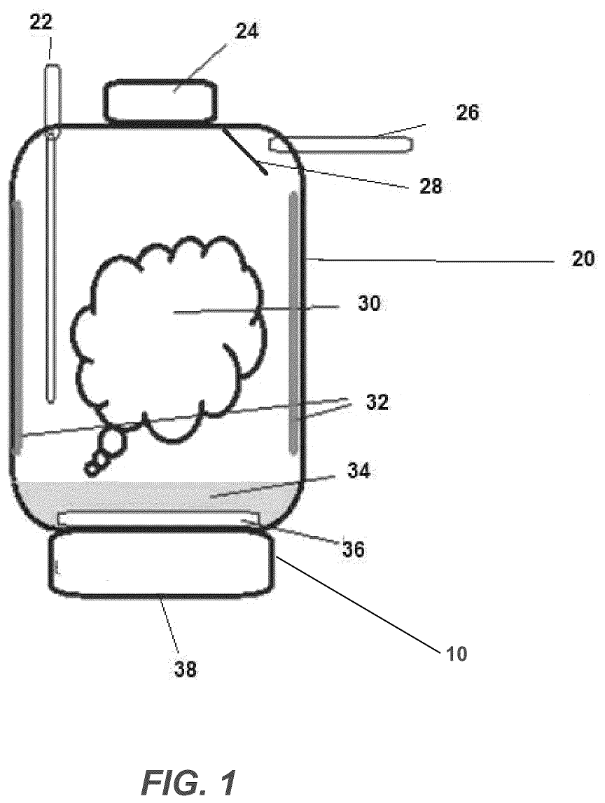

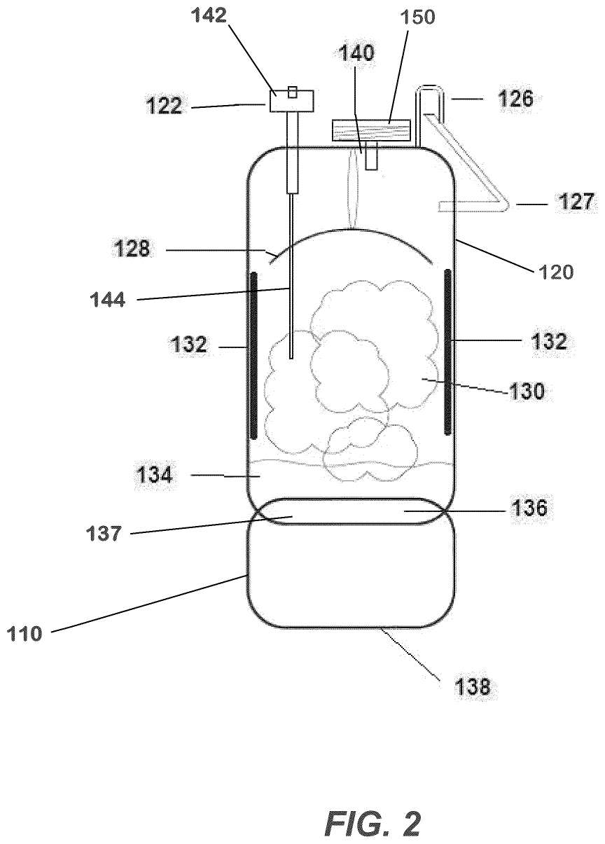

FIG. 1 shows a schematic cross section of an embodiment of the vaporizer 10 of the invention. The vaporizer 10 of FIG. 1 comprises a central chamber 20 containing an amount of liquid 34 in the lower part of the chamber 20. An ultrasonic head 36 is arranged at the bottom of the chamber 20 and reaches into the liquid 34. In the exemplary embodiment of FIG. 1 the liquid 34 is water containing soluble medicines. Below the ultrasonic head 36 and provided as a socket for the chamber 20 is arranged a battery 38 for supplying electric current to the ultrasonic head and further comprising electronic control elements. Applied at least to parts of the walls of chamber 20 is a heating system 32 for increasing the temperature of vapor 30 generated by means of the ultrasonic head 36 in the chamber 20 above the liquid 34. The upper or top part of the chamber comprises a vapor outlet 26 through which the heated vapor 30 generated in the chamber 20 can be directed to a user. The vapor outlet 26 is desirably in the form of a smoking pipe, such as a pipestem or mouthpiece, and the user will typically puff on the smoking pipe to remove an amount of vapor 30 thereby introducing the vapor 30 into the user's pulmonary system. An air inlet 22 for supplying the chamber with ambient air for pressure equalization when the vapor 30 is generated and removed from the chamber 20 is arranged at the top of the chamber 20. The air inlet 22 is typically an elongate tube reaching into the chamber 20 to a position in proximity to the liquid 34 so that air entering the chamber 20 via the air inlet 22 is not short circuited to the vapor outlet 26. A screen 28 functioning as a droplet trap is provided in the vicinity of the vapor outlet 26 to prevent larger droplets from entering the vapor outlet 26. A reservoir 24 is provided on the top side of the chamber 20 for containing medicines to be introduced in the chamber, either into vapor 30 or into the liquid 34, for administering such medicines via the vapor 30 to the user.
FIG. 2 shows a schematic cross section of a second embodiment of the vaporizer of the invention. The vaporizer of FIG. 2 comprises a central chamber 120 containing an amount of liquid, which is an aqueous solution 134 containing, e.g. salt(s), buffering agent(s), drug(s) such as one or more NSAIDs and/or nicotine, in the lower part. An ultrasonic head 136 comprising an ultrasonic membrane 137 is arranged at the bottom of the chamber. The ultrasonic membrane is in contact with the aqueous solution 134 to be vaporized. Below the ultrasonic head 136 and provided as a socket for the chamber is arranged a battery 138 for supplying electric current to the ultrasonic head and further comprising electronic control elements. Applied at least to parts of the chamber walls are heating elements 132. If chamber 120 is a cylinder, the heating elements 132 may be circumferentially provided in a section of the chamber wall. Heating elements 132 increase the temperature of vapor 130 generated by means of the ultrasonic membrane 137 in the chamber 120 above the aqueous solution 134. The upper or top part of the chamber 120 comprises a vapor outlet 126, through which the heated vapor generated in the chamber can be directed to a user. The vapor outlet 126 is desirably in the form of a smoking or aspiration pipe, such as a pipestem or mouthpiece, and the user will typically puff on the smoking or aspiration pipe to remove an amount of vapor 130 from the chamber 120 thereby introducing the vapor 130 into the user's pulmonary system. Vapor outlet 126 has a closure 127 to close the vapor outlet 126 when not in use. An air inlet 122 for supplying the chamber 120 with ambient air for pressure equalization when the vapor 130 is generated and removed from the chamber 120 is arranged at the top of the chamber 120 and comprises an air inlet valve 142 which is a check valve or one-way valve which opens only when the pressure inside the vapor chamber 120 is lower than the ambient pressure outside the vapor chamber 120. The air inlet 122 has an elongate tube 144 reaching into the chamber 120 to a position in proximity to the liquid 134. The elongate tube 144 minimizes short circuiting of air entering via the air inlet 122 to the vapor outlet 126, which would reduce the dosage of vapor 130 to the vapor outlet 126. A screen 128 is provided to act as a droplet trap. Screen 128 is a centrally located screen in the upper part of chamber 120 below the vapor outlet 126. Screen 128 prevents larger droplets from entering the vapor outlet 126. An access opening 140 is provided on the top side of the chamber 120 for reversibly closing the chamber 120 which enables that medicines and/or other additives can be introduced into the chamber 120, either into vapor or into the liquid, for administering such medicines and/or other additives via the vapor to the user. Once the medicines and/or additives have been introduced into the chamber 120 the access opening 140 is closed by a typical closing mechanism such as a screw cap or press-in plug.

In the present disclosure the term “vapor” is also denoted as “fog” and these terms may be used interchangeably for describing the aerosol generated from a liquid inside the vaporizer by the means for cold vaporization of the liquid.

In the vaporizer 10, 110 according to the invention, a vapor chamber 20, 120 is reversibly sealed against the outer environment. In other words, the chamber is air (i.e. gas) and liquid tight and/or reversibly air tight and reversibly liquid tight. The terms “reversibly sealed”, “reversibly airtight”, “reversibly gas tight”, “reversibly liquid tight” mean that the chamber can be opened and closed as convenient, e.g. for cleaning or maintenance purposes or for introducing the liquid to be vaporized, and closed thereafter, and when it is in its closed, the chamber is, except for the air inlet and vapor outlet, sealed from the outer environment.

The air inlet 22, 122 is designed such that ambient air is introduced into the chamber when the pressure in the chamber is lower than the ambient pressure outside the chamber. The air inlet 22, 122 may be designed such that the air is introduced actively into the chamber under said condition. This may be embodied by an air transportation system combined with pressure sensors measuring the ambient pressure and the pressure in the vapor chamber 20, 120 in combination with means comparing the pressure measured by the sensor outside the chamber 20, 120 with the pressure measured inside the chamber such that, when the pressure measured outside the chamber is greater than the pressure measured inside the chamber, the air transportation system causes air to enter the chamber as long as the pressure inside the chamber reaches the pressure outside the chamber. The air transportation system can be embodied e.g., by pumping means or suction means. In other embodiments of the invention ambient air is introduced passively when the ambient pressure outside the chamber is greater than the pressure inside the chamber, preferably by a check valve or one-way valve that opens exclusively when the ambient pressure outside the chamber is greater than the pressure inside the chamber.

In preferred embodiments of the invention, the vaporizer 10, 110 further comprises means for reversibly closing the vapor outlet 26, 126 preferably when the vaporizer is not in use by a user. The means for reversibly closing the vapor outlet can be e.g., a one-way valve provided in the lumen of the vapor outlet exclusively when the user sucks on the vapor outlet 26, 126. In other embodiments, the vapor outlet 26, 126 can be reversibly closed by a closing mechanism covering the outside opening of vaper outlet.

The term “cold vaporization” means that the liquid is vaporized essentially without the use of heat elements. Preferred means for cold vaporization of the liquid are means for emitting ultrasonic waves into the liquid such as an ultrasonic head 36, 136 which is also synonymously referred to herein as “ultrasonic element”. In preferred embodiments of the invention, the ultrasonic head comprises a membrane 137 for generating ultrasonic waves for vaporization of the liquid. In other preferred embodiments, the ultrasonic head comprises one or more reverse piezoelectric elements (i.e. excited crystals generate ultrasonic waves due to reverse piezoelectric effect) generating ultrasonic waves causing vaporization of the liquid. Vaporization by means of reverse piezoelectric elements is especially useful when included as means for cold vaporization in the inventive e-cigarette since the reverse piezoelectric elements are highly suited for miniaturization.

It is preferred according to the invention that the liquid is in direct contact with the ultrasonic waves-emitting element, preferably the ultrasonic head 36, 136.

Cold vaporization, in particular by ultrasonic waves emitted from an ultrasonic elements leads to a relatively cold vapor which can be problematic for the user inhaling the cold vapor which frequently leads at least to an uncomfortable feeling (and, thus, to low user friendliness and low user acceptance of the vaporizer), but may also lead to bronchial constriction which would be highly problematic for users suffering from pulmonary conditions. In particular, bronchial constriction would be a serious problem for subjects having COPD or pre-COPD conditions such as chronic bronchitis.

To circumvent the above-described problems, the vaporizer of the invention comprises means 32, 132 for heating vapor generated in the chamber are attached to at least a part of the wall(s) of the chamber or integrated in at least a part of the wall(s) of the chamber. The heating means 32, 132 preferably are designed and, more particularly, regulated such that the vapor is heated to a temperature of about less than 100° C., preferably from about ambient temperature such as about 20, about 21, about 22, about 24 or about 25° C. to about less than 90°C., more preferably about 30 to about 80° C., most preferably about 30° C. to about 40° C.

In preferred embodiments, the chamber 20, 120 of the vaporizer according to the present invention contains a liquid 34, 134 to be vaporized by the means for cold vaporization. Preferably, the liquid 34, 134 is water or an aqueous solution, suspension or emulsion. In preferred embodiments, the liquid, preferably the aqueous solution, suspension or emulsion, comprises one or more additives such as nicotine and/or one or more drugs and/or one or more flavors. Preferred additives are selected, for example, from bactericidal and virucidal drugs, salts such as NaCl, KCl,, LiCl and mixtures thereof, amino acids such as arginine, and buffering agents such as NaHCO₃. In certain embodiments of the invention the composition comprises a stimulant. A preferred stimulant is nicotine. Further preferred additives for use in the invention include, but are not limited to, non-steroidal anti-inflammatory drugs (NSAIDs) such as ibuprofen, naproxen, flurbiprofen, ketoprofen, diclofenac, diflunisal, etodolac, fenoprofen, indomethacin, meclofenamate, mefenamic acid, meloxicam, nabumetone, oxaprozin, piroxicam, sulindac, tolmetin, celecoxib, acetylated salicylates, non-acetylated salicylates, and combinations thereof. Especially preferred NSAIDs for use in the invention are ibuprofen and naproxen and mixtures thereof, most preferably ibuprofen.

Especially preferred liquid compositions for use in the present invention are hypertonic solutions of NSAIDs, such as compositions disclosed in US 2018/0296509 A1, the contents of which are hereby incorporated in its entirety by reference, such as described in paragraph [0023] thereof.

Particularly preferred compositions for use in the invention are bactericidal and virucidal compositions characterized by comprising a NSAID at a concentration of from about 5 and to about 500 mM solubilized in saline solution, preferably a hypertonic saline solution comprising a concentration of from about 0.3 to about 2 Molar NaCl; more preferably from about 0.4 and 1.1 M NaCl; even more preferably from about 0.9 to about 1.05 M NaCl. More preferably, the concentration of NSAID is in the range from about 5 to about 180 mM; more preferably less than about 180 mM; even more preferably from about 5 to about 50 mM. Preferably, the NSAID is selected from those as outlined above (including mixtures thereof), most preferably ibuprofen. Preferred counterions are monovalent cations selected from sodium, potassium, lithium and combinations thereof. Preferably, aqueous compositions, in particular aqueous solutions, for use in the invention have pH of from about 6.0 to about 8.5; preferably from about 7.0 to about 8.0; more preferably from about 7.5 to about 7.9.

In preferred embodiments, the vaporizer according to the invention comprises one or more reservoirs 24 designed to contain one or more substances (also denoted herein as “additives”) to be introduced into the chamber, the reservoir having a reversibly closeable connection to the chamber. In one embodiment, the reservoir 24 is designed to contain the liquid to be vaporized in the chamber. A reservoir 24 designed for containing the liquid is also denoted herein as “liquid reservoir”. In other embodiments, at least one reservoir 24 is designed to contain one or more additives such as nicotine and/or drugs and/or flavors to be introduced in the chamber. A reservoir designed for containing additive(s) is also denoted herein as “additive reservoir”. Preferably, an additive reservoir 24 is arranged in the vaporizer 10, 110 such that the additive(s) are introduced into the vapor 30, 130 generated in the chamber. In other embodiments, the vaporizer 10, 110 may comprise (instead of a reservoir or additionally thereto) an access opening 140 for introducing one or more substances into the vapor chamber 20, 120. An access 140 opening is closable with a screw-in plug 150 or a snap-in plug or other similar closure.

In certain embodiments of the invention the vaporizer 10, 110 further comprising means for hindering liquid droplets from entering the vapor outlet, such as the droplet trap 28, 128.

The vaporizer of the invention preferably further comprises means for supplying the cold vaporization means with electric current. The electric current supplying means are preferably embodied by a battery or accumulator 38, 138, preferably a rechargeable battery or rechargeable accumulator. The vaporizer of the invention preferably comprises means for controlling the means for cold vaporization of a liquid, preferably the one or more ultrasonic heads 36, 136.

The vaporizer 10, 110 of the invention may preferably be provided in the form of and having the dimensions of an electric cigarette.

Thus, the present invention also relates to an e-cigarette comprising at least the essential components of the vaporizer as defined herein. Preferred embodiments of the e-cigarette according to the invention have already been described above.

The invention also relates to a method of treatment of COPD or a pre-COPD condition such as chronic bronchitis in a subject in need comprising administration of a vapor generated from a liquid as defined herein by means of the vaporizer and/or e-cigarette of the invention.

The present invention is further described with reference to drawings showing schematic representations of embodiments of the vaporizer of the invention.

What is claimed is:

1. A vaporizer comprising
a vapor chamber designed to contain vapor generated from a liquid inside the chamber under temperature controlled condition;
an air inlet designed for actively or passively introducing ambient air into the chamber under the condition that the pressure inside the chamber is lower than the ambient pressure outside the chamber;
a vapor outlet for discharging vapor generated in the chamber and directing the vapor to a user;
one or more means for cold vaporization of a liquid; and
heating means for heating vapor generated in the chamber.

2. The vaporizer of claim 1 wherein the means for cold vaporization of a liquid is one or more ultrasonic heads.

3. The vaporizer of claim 1 wherein the means for heating vapor generated in the chamber are attached to at least a part of the wall(s) of the chamber or integrated in at least a part of the wall(s) of the chamber.

4. The vaporizer of claim 1 wherein the air inlet comprises a one way valve exclusively opening when the pressure inside the chamber is lower than the ambient pressure outside the chamber.

5. The vaporizer of claim 1 further comprising means for reversibly closing the vapor outlet.

6. The vaporizer of claim 1 wherein the chamber contains a liquid to be vaporized by the means for vaporization.

7. The vaporizer of claim 6 wherein the liquid is water or an aqueous solution.

8. The vaporizer of claim 6 wherein the liquid comprises one or more additives.

9. The vaporizer of claim 8 wherein the additive(s) is/are selected from nicotine, drugs, salts, buffering agents and flavors.

10. The vaporizer of claim 1 further comprising a reservoir designed to contain a substance or substances to be introduced into the chamber, the reservoir having a reversibly closable connection to the chamber.

11. The vaporizer of claim 1 further comprising means for hindering liquid droplets from entering the vapor outlet.

12. The vaporizer of claim 1 provided in the form of and having the dimensions of an electronic cigarette.

13. A vaporizer comprising
a vapor chamber having a lower part and an upper part and sidewalls;
an aqueous solution having one or more additives contained in the lower part of the chamber;
one or more ultrasonic heads provided in the lower part of the vapor chamber for cold vaporization of the aqueous solution to form a vapor;
one or more electrical resistance heating elements provided on the sidewalls of the vapor chamber to heat vapor in the vapor chamber;
electronic control elements and a battery for activating and providing power to the one or more ultrasonic heads and the one or more electrical resistance heating elements;
a vapor outlet provided in the upper part of the vapor chamber for discharging vapor formed in the chamber and having a mouthpiece for directing the vapor to a user; and
an air inlet provided in the upper part of the vapor chamber having a one-way inlet valve and an elongate tube extending into the lower part of the vapor chamber for introducing ambient air into the chamber when pressure inside the chamber is lower than ambient air pressure outside the chamber.

14. The vaporizer of claim 13 wherein the additives are selected from the group consisting of nicotine, drugs, salts, buffering agents and flavors.

15. The vaporizer of claim 14 further comprising a reservoir, which is removably attached to the chamber, which contains the aqueous solution.

16. The vaporizer of claim 14 further comprising an access opening and a closure provided in the upper part of the vapor chamber.

17. The vaporizer of claim 14 further comprising a screen located in the upper part of the vapor chamber to trap droplets contained in the vapor and prevent release of the droplets from the vapor outlet.

18. The vaporizer of claim 14 further comprising a vapor outlet closure.
